(19) United States
(12) Patent Application Publication
(10) Pub. No.: US 2010/0062498 A1
(43) Pub. Date: Mar. 11, 2010

(54) POLYPEPTIDE BEING CAPABLE OF INCREASING THE PRODUCTION OF L-METHIONINE, A MICROORGANISM THAT OVEREXPRESSES SAID POLYPEPTIDE AND A PROCESS OF PREPARING L-METHIONINE IN HIGH YIELD USING SAME

(75) Inventors: Young Hoon Park, Gyeonggi-do (KR); Kwang Myung Cho, Gyeonggi-do (KR); So Young Kim, Gyeonggi-do (KR); Yong Uk Shin, Gyeonggi-do (KR); Hye Won Um, Gyeonggi-do (KR)

Correspondence Address: KNOBBE MARTENS OLSON & BEAR LLP, 2040 MAIN STREET, FOURTEENTH FLOOR, IRVINE, CA, 92614, US

(73) Assignee: CJ CHEILJEDANG CORPORATION, SEOUL (KR)

(21) Appl. No.: 12/521,541

(22) PCT Filed: Dec. 28, 2007
(86) PCT No.: PCT/KR07/06979

§ 371 Date: Jun. 26, 2009

(30) Foreign Application Priority Data
Dec. 28, 2006 (KR) 10-2006-0136919

(51) Int. Cl. C12P 13/12; C07K 2/00; C07H 21/04; C12N 15/74; C12N 1/21

(52) U.S. Cl. 435/113; 530/300; 536/23.7; 435/320.1; 435/252.3; 435/252.31; 435/252.33; 435/252.34; 435/252.35

(57) ABSTRACT

The present invention relates to a polypeptide capable of increasing the production of L-methionine in a microorganism. In particular, the present invention relates to an YgaZ and YgaH polypeptide or a complex thereof, referred to herein as YgaZH polypeptide, which are novel putative L-methionine exporters, polynucleotides encoding the same, a recombinant vector comprising the polynucleotide, a microorganism transformed with the recombinant vector, and a method for producing L-methionine and/or S-adenosyl-methionine, comprising the steps of culturing the transformed microorganism to produce L-methionine and/or S-adenosyl-methionine, and isolating L-methionine and/or S-adenosyl-methionine. The transformed microorganism of the present invention produces L-methionine in a high yield, thereby being used for medicinal and pharmaceutical industries and feed industry, in particular, animal feeds

### TECHNICAL FIELD

The present invention relates to a polypeptide capable of increasing the production of L-methioninein a microorganism. In particular, the present invention relates to an YgaZ and YgaH polypeptide or a complex thereof, referred to herein as a YgaZH polypeptide, which are novel putative L-methionine exporters, polynucleotides encoding the same, a recombinant vector comprising the polynucleotide, a microorganism transformed with the recombinant vector, and a method for producing L-methionine and/or S-adenosyl-methionine, comprising the steps of culturing the transformed microorganism to produce L-methionine and/or S-adenosyl-methionine, and isolating L-methionine and/or S-adenosyl-methionine.

### BACKGROUND ART

L-methionine is an essential amino acid found in most proteins, and in its unaltered form is an ingredient of soy sauce. Further, L-methionine has been widely used as an animal feed and food additive, as well as a component of medical aqueous solutions and other raw material for medicinal products. Methionine is an important amino acid involved in biochemical methyl transfer reactions, in which a condensation of ATP and methionine yields δ-adenosylmethionine, and δ-adenosylmethionine serves as a universal methyl donor to a variety of acceptors, and is then broken down into cysteine via homocysteine and cystathionine. Neurosporasynthesizes methionine from cysteine. The odor of fermented foods such as soy sauce and cheese is generally due to aldehyde, alcohol, and/or ester generated from methionine.

Further, methionine acts as a precursor of choline, lecithin, creatine or the like, and as a sulfur donor to be used as a raw material for the synthesis of cysteine and taurine. In addition, it is involved in the synthesis of various neurotransmitters in the brain. Methionine and/or S-adenosyl-L-methionine (SAM) is/are also found to prevent lipid accumulation in the liver and arteries and to be effective for the treatment of depression, inflammation, liver diseases and muscle pain.

As summarized below, methionine and/or S-adenosyl-L- methionine has been found, thus far, to have in vivo functions of: 1) preventing lipid accumulation in the liver, where lipid metabolism is mediated, and in arteries to maintaining blood flow to the brain, the heart and the kidneys (Jeon B. R., et al, J. Hepatol., 34(3):395-401, 2001) 2) faciliating digestion, detoxifying and excreting harmful agents, and scavenging heavy metals such as lead 3) acting as an excellent antidepressant when methionine is administered at a daily dose of 800-1,600 mg (Mischoulon D., et al., Am. J. Clin. Nutr., 76(5):1158S-61S, 2002) 4) improving liver functions against liver diseases (Mato J. M., FASEB J., 16(1):15-26, 2002), particularly, attenuating alcohol-induced liver injury (Rambaldi A., Cochrane Database Syst. Rev., 4: CD002235, 2001) 5) showing an anti-inflammation effect versus osteoarthritis and promoting the healing of joints (Sander O., ACP J. Club. 138(1): 21, 2003; Soeken K. L., et al, J. Fam. Pract., 51(5): 425-30, 2000) 6) acting as an essential nutrient of hair to prevent brittle hair and depilation (Lackwood D. S., et al., Audiol Neurotol., 5(5):263-266, 2000).

Methionine can be produced by chemical and biological synthesis for the application to foods including animal feeds and medicine.

On the whole, chemical synthesis for the production of methionine utilizes the hydrolysis of 5-(β-methylmercaptoethyl)-hydantoin. However, the chemical synthesis suffers from the problem of synthesizing methionine in a mixture of L- and D-forms.

In the biological route, advantage is taken of the proteins involved in methionine production. Biosynthesis of L-methionine is achieved from homoserine with the aid of enzymes encoded by metA, metB, metC, metE, and metH genes. In detail, homoserine succinyltransferase, which is the first enzyme in a methionine biosynthesis pathway and encoded by metA, functions to convert homoserine into O-succinyl-L-homoserine. Subsequently, O-succinyl-L-homoserine is converted into cystathionine by O-succinylhomoserine lyase which is encoded by metB. Cystathionine beta lyase which is encoded by metC is responsible for the conversion of cystathionine into L-homocystein. Two enzymes, cobalamin-independent methionine synthase and cobalamin-dependent methionine synthase, which are respectively encoded by metE and metH, function to synthesize N(5)-methyltetrahydrofolate (5-MTHF) that acts as the methyl donor necessary for the synthesis of L-methionine from L-homocysteine.

In the biological route, L-methionine is synthesized through a series of intimative reactions catalyzed by the enzymes. Thus, these enzymes and proteins controlling them may be genetically modified for improving and controlling L-methionine synthesis. For example, Japanese Pat. Laid-Open Publication No. 2000-139471 disclosesan L-methionine production method using Escherichia sp. in which metBL is overexpressed in the presence of a leaky type of metK, with thrBc and metJ eliminated. US 2003/0092026 A1 describes a Corynerbacterium sp. that is modified to remove metD, a factor inhibitory to L-methionine synthesis, therefrom. US 2003/0088886 discloses that the production of L-methionine can be improved by increasing the expression of methionine synthase and cystathionine γ-synthase, which are respectively encoded by metA and metB, in a transgenic plant.

When L-methionine is synthesized at a certain level or higher, it inhibits its own further production via a feedback loop. Therefore, for the high expression of L-methionine, it is very important to export the synthesized L-methionine. L-methionine exporters of L-methionine that is synthesized through a series of intimative reactions are disclosed in several literatures. For example, DE 10,305,774 Al discloses an L-methionine production method using Escherichia coli, in which the L-methionine exporter, YjeH protein is overexpressed, with the metJ gene eliminated, so as to produce 0.8 g/L of L-methionine. Further, reported are BrnF and BrnE polypeptides, which are L-methionineexporters of Corynebacterium glutamicum (C. Troschel, et al, Journal of Bacteriology, p.3786-3794, June 2005).

Accordingly, in order to produce L-methionine in a high yield through a biological route, the present inventors have made an effort to explore L-methionine exporters. They found that L-methionine can be produced in a high yield by overexpressing an YgaZH polypeptide, which is a complex of YgaZ and YgaH encoded by ygaZ and ygaH genederived from Escherichia coli and their base sequences are disclosed, but their functions not yet, thereby completing the present invention.

### DISCLOSURE OF INVENTION

### Technical Problem

It is an object of the present invention to provide an YgaZ and YgaH polypeptide or a complex thereof, referred to herein as a YgaZH polypeptide, capable of increasing the production of L-methionine in microorganism, and polynucleotides encoding the same.

It is another object of the present invention to provide a recombinant vector comprising the polynucleotide.

It is still another object of the present invention to provide a microorganism producing L-methionine in a high yield, transformed with the recombinant vector.

It is still another object of the present invention to provide a method for producing L-methionine and/or S-adenosyl-methionine, comprising the steps of culturing the transformed microorganism to produce L-methionine and/or S-adenosyl-methionine and isolating L-methionine and/or S-adenosyl-methionine.

### BRIEF DESCRIPTION OF THE DRAWINGS

FIG. 1 illustrates the construction of a recombinant plasmid pCL-(trc)ygaZH, comprising DNA encoding L-methionine exporters of the present invention.

### BEST MODE FOR CARRYING OUT THE INVENTION

In one embodiment, the present invention provides an YgaZ and YgaH polypeptide or a complex thereof, referred to herein as a YgaZH polypeptide, which are putative L-methionine exporters improving the production of L-methionine in a microorganism.

Most preferably, amino acid sequences of the YgaZ polypeptide and YgaH polypeptide are represented by SEQ ID NOs. 1 and 2, respectively. It is apparent to those skilled in the art that a polypeptide having an amino acid sequence having 80% or more homology with SEQ ID NO. 1 or 2 and having an activity of exporting the produced L-methionine is preferably included within the scope of the invention, and a polypeptide having an amino acid sequence having 90% or more homology with SEQ ID NO. 1 or 2 and improving the production of L-methionine in a microorganism is more preferably included within the scope of the invention.

The term “polypeptide having an amino acid sequence having 80% or more homology with SEQ ID NO. 1 or 2” refers to a polypeptide, in which 1 to 50 amino acids of SEQ ID NO. 1 or 2 are modified by known methods in the art such as deletion, substitution, insertion and addition, and has an activity of exporting L-methionine produced in a microorganism.

In one embodiment, the present invention further provides a polynucleotide that encodes an YgaZ and YgaH polypeptide, or a complex thereof, referred to herein as a YgaZH polypeptide, capable of improving the productivity of L-methionine in the microorganism.

Preferably, the polynucleotide may be a base sequence encoding the YgaZ polypeptide having amino acid sequences represented by SEQ ID NO. 1 and the YgaH polypeptide represented by SEQ ID NO. 2. More preferably, the polynucleotide encoding the polypeptide having amino acid sequences represented by SEQ ID NO. 1 may be represented by SEQ ID NO. 3, and the polynucleotide encoding the polypeptide represented by SEQ ID NO. 2 may be represented by SEQ ID NO. 4.

In one embodiment, the present invention further provides a recombinant vector comprising the polynucleotide encoding the polypeptide of SEQ ID NO. 1 and the polynucleotide encoding the polypeptide of SEQ ID NO. 2. Preferably, the recombinant vector may comprise the polynucleotide represented by SEQ ID NO. 3 and the polynucleotide represented by SEQ ID NO. 4, or each polynucleotide represented by SEQ ID NO. 3 or 4. The recombinant vector may be pCL-(trc)ygaZH fabricated according to specific Examples of the present invention.

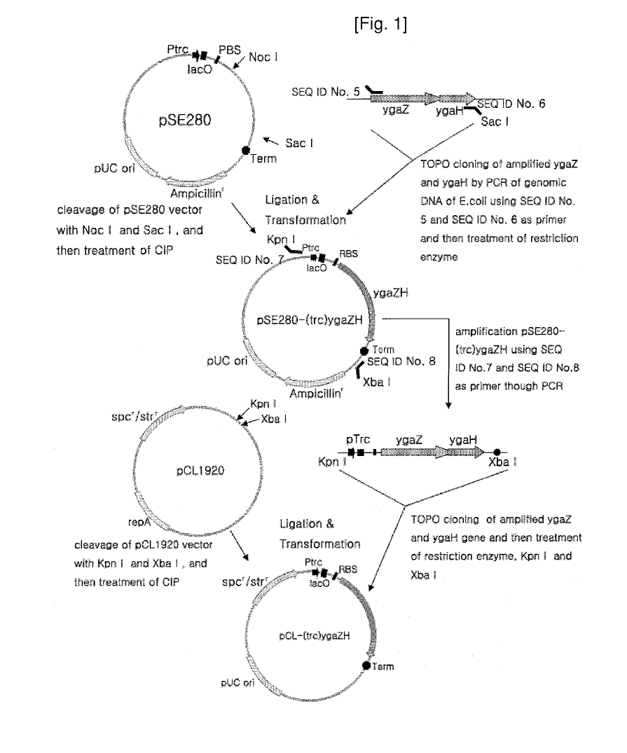

The recombinant vector can be easily fabricated by those skilled in the art according to any known method using recombinant DNA techniques. In a specific embodiment of the present invention, ygaZ and ygaH genes present as an operon in the genomic DNA of the microorganism are co-amplified by PCR to obtain an ygaZH gene, and the product is cloned into a cloning vector to obtain a cloning product containing the ygaZH gene. Then, the cloning product is introduced into an expression vector to fabricate an expression vector containing the ygaZH gene. In this connection, “a suitable regulatory sequence” regulating the transcription and translation of the ygaZH gene can be introduced into the expression vector. In one embodiment of the present invention, a promoter of the ygaZH gene in the vector is replaced with a trc promoter, and then the ygaZH gene containing the trc promoter is introduced into the expression vector to fabricate a recombinant vector pCL-(trc)ygaZH (FIG. 1).

In the production method of the recombinant vector, any known cloning vector may be used, preferably apCR2.1-TOPO vector. Further, the microorganism may be Escherichia coli, preferably Escherichia coli W3110.

The term “vector” as used herein refers to an extra chromosomal element capable of carrying a nonessential gene for cell metabolism, and usually in the form of circular double-stranded DNA molecules. The term “element” as used herein refers to a self-replicating sequence, a genome insertion sequence, a phage or nucleotide sequence, a linear or circular, single- or double-stranded DNA or RNA. Generally, the vector contains a suitable transcription or translation regulatory sequence, a selection marker, and a competent sequence for self-replication or chromosome insertion. A suitable vector includes a 5-region of a DNA fragment that regulates transcription initiation and a 3-region of a DNA fragment that controls transcription termination. The term “suitable regulatory sequence” indicates a sequence that regulates the transcription and translation of the above polynucleotide. Examples of the regulatory sequence include a ribosomal binding sequence (RBS), a promoter, and a terminator. As used herein, the promoter is not particularly limited provided that it is a sequence that drives initiation of transcription of polynucleotide encoding SEQ ID NOs. 1 and 2, which are the polypeptides having an activity of exporting the L-methionine produced in the microorganism, and examples thereof may include CYC1, HIS3, GAL1, GAL10, ADH1, PGK, PHO5, GAPDH, ADC1, TRP1, URA3, LEU2, ENO, TPI (useful for expression in Saccharomyces), lac, trp, λP_{L}, λP_{R}, T7, tac and trc(useful for expression in E. coli). Further, the terminator region may be derived from various genes of a preferred host cell and may be optionally omitted.

In one embodiment, the present invention provides a microorganism transformed by introducing the recombinant vector into the microorganism. In a specific embodiment, the host microorganism may be one selected from the group consisting of Escherichia, Aerobacter, Schizosaccharomyces, Zygosaccharomyces, Pichia, Kluyveromyces, Candida, Hansenula, Debaryomyces, Mucor, Torulopsis, Methylobacter, Salmonella, Bacillus, Streptomyces, Pseudomonas and Corynebacterium (species), preferably Escherichia coli, and more preferably E. coli W3110 or E. coli MF001, which is the non-methionine auxotrophic threonine-producing strain.

In a specific embodiment, the present invention provides a microorganism transformed by introducing the recombinant vector pCL-(trc)ygaZH into E. coli W3110 (accession number: KCCM10818P) and a microorganism transformed by introducing the recombinant vector pCL-(trc)ygaZH into the non-methionine auxotrophic threonine-producing strain, E. coli MF001.

The transformed microorganisms can be easily prepared by those skilled in the art according to any known method. Transformation artificially generates genetic alteration by introducing a foreign DNA into a cell, and examples thereof include a CaCl₂ method, a Hanahan method that is an improved CaCl₂ method by using DMSO (dimethyl sulfoxide) as a reducing material, and electroporation. In embodiments of the present invention, the transformed microorganism is prepared by introducing the recombinant vector pCL-(trc)ygaZH into the host microorganism using electroporation.

In one embodiment, the present invention provides a method for producing L-methionine, comprising the step of culturing the transformed microorganism that is prepared by introducing the recombinant vector into the microorganism, preferably the transformed microorganism (accession number: KCCM10818P) that is prepared by introducing the recombinant vector pCL-(trc)ygaZH into E. coli W3110, or culturing the transformed microorganism that is prepared by introducing the recombinant vector pCL-(trc)ygaZH into the non-methionine auxotrophic threonine-producing strain, E. coli MF001. Specifically, the present invention provides a method for producing L-methionine, comprising the steps of (a) culturing the transformed microorganism; (b) concentrating L-methionine in the broth or microorganisms; and (c) separating residual L-methionine and any constituent of the fermentation broth and/or the biomass. In the production method of L-methionine, the transformed microorganism of step (a) is accession number: KCCM10818P or the transformed microorganism by introducing the recombinant vector pCL-(trc)ygaZH into the non-methionine auxotrophic threonine-producing strain, E. coli MF001.

In the production method of L-methionine of the present invention, the cultivation of the transformed microorganism overexpressing L-methionine may be conducted in suitable media and under culture conditions known in the art. According to strains used, the culturing procedures can be readily adjusted by those skilled in the art. Examples of the culturing types include batch culture, continuous culture and fed-batch culture, but are not limited thereto. Various culturing procedures are disclosed in literature, for example, “Biochemical Engineering” (James M. Lee, Prentice-Hall International Editions, pp 138-176, 1991).

The media used in the culture should preferably meet the requirements of a specific strain. Typically culture media contain various carbon sources, nitrogen sources and minerals. Examples of the carbon sources useful in the present invention include carbohydrates such as glucose, fructose, sucrose, lactose, maltose, starch, and cellulose, lipids such as soybean oil, regular sunflower oil, castor oil, and coconut oil; fatty acids such as palmitic acid, stearic acid, and linoleic acid, alcohol such as glycerol and ethanol, and organic acids such as acetic acid. These carbon sources may be used alone or in combination. Examples of nitrogen sources useful in the present invention include organic nitrogen sources such as peptone, yeast extract, broth, malt extract, corn steep liquor (CSL) and soy bean, and inorganic nitrogen sources such as urea (CO(NH₂)₂), ammonium sulfate ((NH₄)₂SO₄), ammonium chloride (NH₄Cl), ammonium phosphate ((NH₄)₂HPO₄), ammonium carbonate ((NH₄)₂CO₃) and ammonium nitrate (NH₄NO₃). These nitrogen sources may be used alone or in combination. To the media, phosphorus sources such as potassium dihydrogen phosphate (KH₂PO₄), dipotassium hydrogen phosphate (K₂HPO₄) or corresponding sodium-containing salts may be added. In addition, the media may contain metal salts such as magnesium sulfate and ferrous sulfate. Further, the media may be supplemented with amino acids, vitamins, and appropriate precursors. These media or precursors may be added to cultures by a batch type or continuous type method.

During cultivation, ammonium hydroxide, potassium hydroxide, ammonia, phosphoric acid, and sulfuric acid may be properly added so as to adjust the pH of the cultures. Defoaming agents such as fatty acid polyglycol ester may be properly added so as to reduce the formation of foams in cultures. To maintain the cultures in aerobic states, oxygen or oxygen-containing gas (e.g., air) may be injected into the cultures. The cultures are maintained at 20 to 45 C and preferably at 25 to 40 C. The cultivation may be continued until a desired amount of L-methionine is obtained, and preferably for 10 to 160 hrs.

The isolation of L-methionine from the culture broth can be performed by the conventional method known in the art. Examples thereof may include centrifugation, filtration, ion-exchange chromatography, and crystallization. For example, the cultures are subjected to low-speed centrifugation to remove the biomass, and the supernatant was separated by ion-exchange chromatography.

As described in the following Examples, it was found that the transformed microorganism (accession number: KCCM10818P) that is prepared by introducing the recombinant vector pCL-(trc)ygaZH into E. coli W3110, or the transformed microorganism that is prepared by introducing the recombinant vector pCL-(trc)ygaZH into the non-methionine auxotrophic threonine-producing strain E. coli MF001 is a strain producing L-methionine in a high yield.

The methionine produced according to the present invention is an L-form. The L-form is synthesized in vivo, and readily utilized in organisms. Therefore, the L-form has an advantage over the D-form. L-methionine finds applications in various industries, such as an additive for feed and foodstuff, a medicinal material, a sulfur source, and a medicine. In one important metabolism pathway, L-methionine is adenosylated to form S-adenosyl-methionine. Thus, as the biosynthesis of L-methionine actively occurs, the amount of its important metabolite S-adenosyl-methionine also increases.

In one embodiment, the present invention further provides a method for producing S-adenosyl-methionine, comprising the step of culturing the transformed microorganism capable of producing S-adenosyl-methionine in a high yield, which is prepared by introducing the recombinant vector into the host microorganism, preferably the transformed microorganism (accession number: KCCM10818P) that is prepared by introducing the recombinant vector pCL-(trc)ygaZH into E. coli W3110, or culturing the transformed microorganism that is prepared by introducing the recombinant vector pCL-(trc)ygaZH into the non-methionine auxotrophic threonine-producing strain, E. coli MF001. Specifically, the present invention provides a method for producing S-adenosyl-methionine, comprising the steps of (a) culturing the transformed microorganism; (b) concentrating S-adenosyl-methionine in the broth or microorganisms; and (c) separating residual S-adenosyl-methionine and any constituent of the fermentation broth and/or the biomass.

### Mode for the Invention

A better understanding of the present invention may be obtained through the following examples which are set forth to illustrate, but are not to be construed as the limit of the present invention.

### Example 1

### Construction of Recombinant Vector Containing ygaZH Gene of E. coli

In the present invention, an ygaZ gene that encodes a hypothetical protein and an ygaH gene that encodes a putative transport protein in Escherichia coli were replaced with a trc promoter, respectively. Then, vectors containing the genes were prepared. The ygaZ and ygaH genes are present as anoperon in Escherichia coli. The base sequences of the genes are known, but their functions are not yet identified. It has been known that the ygaZ gene has738 bp, and ygaH gene has 336 bp.

### Example 1-1

### Amplification of ygaZH Gene

The genomic DNA (gDNA) was extracted from Escherichia coli W3110 strain using a Genomic-tip system (QIAGEN, hereinafter the same). In order to co-amplify the ygaZ and ygaH genes that are present as an operon, polymerase chain reaction (hereinafter, abbreviated to “PCR”) was performed using the gDNA as a template and an Accur Power HL-PCR Premix (BIONEER Co., hereinafter the same). At this time, a pair of oligonucleotides having the base sequences represented by SEQ ID NOs. 5 and 6 was used as primers. Each of the primers having the base sequences represented by SEQ ID NOs. 5 and 6 contained the recognition sites of Nco and Sac. The primer represented by SEQ ID NO. 5 contained a base sequence of 5′-region of the ygaZ gene, and the primer represented by SEQ ID NO. 6 contained a base sequence of 3′-region of the ygaH gene.

10 ng of template and each 100 pmole of oligonucleotides represented by SEQ ID NOs. 5 and 6 were mixed with Accur Power HL-PCR Premix to prepare 20□ of reaction solution, and then PCR was performed under the conditions including 30 cycles of denaturation at 94° C. for 30 sec, annealing at 52° C. for 30 sec and elongation at 72° C. for 1 min 30 sec.

The PCR product was subjected to electrophoresis on an agarose gel, and then a

DNA band having the desired size of 1071 by was found (result not shown). The band was isolated and purified, and then cloned into a cloning vector pCR2.1 using a TOPO TA Cloning kit (Invitrogen, USA, hereinafter the same).

### Example 1-2

### Introduction of ygaZH gene into Expression Vector

The pCR2.1 vector carrying the PCR product prepared in Example 1-1 was treated with Nco and Sac to cleave the ygaZH gene, and then isolated and purified. Further, the expression vector pSE280 (Invitrogen, USA) was treated with Nco and Sac, and then treated with 30□ of CIP (calf intestinal phosphatase).

1□ of T4 DNA ligase and 1□ of ligase buffer were added to the mixure of 100 ng of the ygaZH gene and10 ng of pSE280 to a total volume of 10□, and then subjected to reaction for 6 hrs at 16° C. After the reaction was terminated, E. coli DH5α were transformed with the resultant vector products and cultured, and then disrupted. The region containing ygaZ and ygaH genes were separated and purified, which was designated as “pSE-(trc)ygaZH”.

### Example 1-3

### Construction of Recombinant Vector Containing ygaZH Gene of which Promoter was Replaced with trc Promoter

In order to prepare the ygaZH gene containing the trc promoter, PCR was performed using DNA of pSE-(trc)ygaZH prepared in Example 1-2 as a template and a pair of oligonucleotides represented by SEQ ID NOs. 7 and 8 as primers to amplify the region from trc promoter to terminator of ygaZH gene. Each of primers represented by SEQ ID NOs. 7 and 8 contained the recognition sites of the restriction enzyme, Kpn and Xba.

10 ng of template and each 100 pmole of oligonucleotides represented by SEQ ID NOs. 7 and 8 were mixed with Accur Power HL-PCR Premix to prepare 20□ of reaction solution, and then PCR was performed under the conditions including 30 cycles of denaturation at 94° C. for 30 sec, annealing at 55° C. for 30 sec and elongation at 72° C. for 2 min 30 sec.

The PCR product was subjected to electrophoresis on an agarose gel, and then a DNA band having the desired size of 2200 by was found. The band was isolated and purified, and then treated with KpnI and XbaI to cleave the ygaZH gene containing the trc promoter, followed by isolation and purification. Further, the expression vector pCL1920 was treated with Kpn and Xba, and then treated with 30□ of CIP(calf intestinal phosphatase).

1□ of T4 DNA ligase and 1□ of ligase buffer were added to the mixture of 100 ng of the ygaZH gene containing the trc promoter and 10 ng of pCL1920 to a total volume of 10□, and then subjected to reaction for 6 hrs at 16° C. After the reaction was terminated, E. coli DH5α were transformed with the resultant vector products and cultured, and then disrupted. The region containing ygaZ and ygaH genes were separated and purified from the transformed E. coli, which was designated as a recombinant plasmid “pCL-(trc)ygaZH”.

### Example 2

### Preparation of transformed Microorganism Producing High Level of L-methionine

### Example 2-1

### Preparation of Transformed Microorganism “E. coli W3110/ygaZH”

The recombinant plasmid pCL-(trc)ygaZH prepared in Example 1-3 was introduced into E. coli W3110 strain by electroporation to prepare a transformed microorganism. Then, the microorganism was smeared on a Lurina-Bertani (hereinafter, abbreviated to “LB”) solid medium (Bacto tryptone 10 g/L, Bacto Yeast extract 5 g/L, sodium chloride 10 g/L, hereinafter the same) containing spectinomycin (50 □/L), followed by incubation at 37° C. overnight. The colonies of transformed microorganism were obtained, which was designated as “E. coli W3110/ygaZH or CA05-0017” in the present invention. Further, the transformed strain was deposited at the Korean Culture Center of Microorganisms (hereinafter, abbreviated to “KCCM”) on Dec. 13, 2006 under accession number KCCM10818P.

### Example 2-2

### Preparation of Transformed Microorganism “E. coli MF001/ygaZH”

The recombinant plasmid pCL-(trc)ygaZH prepared in Example 1-3 was introduced into the non-methionine auxotrophic threonine-producing strain, E. coli MF001 (Korean Patent Publication No. 10-2006-0000774) by electroporation to prepare a transformed microorganism. Then, the microorganism was smeared on a LB solid medium containing spectinomycin (50 □/L), followed by incubation at 37° C. overnight. The colonies of transformed microorganism were obtained, which was designated as “E. coli MF001/ygaZH” in the present invention.

### Example 3

### Comparison of L-methionine Productivity of Transformed Microorganism

The transformed microorganisms, E. coli W3110/ygaZH and E. coli MF001/ygaZH prepared in Example 2 were cultured on LB solid media containing spectinomycin (50 □/L), and then colonies were obtained and selected. Then, the transformed strains were cultured in Erlenmeyer flasks containing methionine titer medium of Table 1, and the L-methionine productivity was compared.

TABLE 1

| Components | Amount (per liter) | |
|---|---|---|
| Glucose | 40 | g |
| Ammonium sulfate | 17 | g |
| KH₂PO₄ | 1 | g |
| MgSO₄•H₂O | 1 | g |
| FeSO₄•H₂O | 5 | mg |
| MnSO₄•H₂O | 5 | mg |
| ZnSO₄ | 5 | mg |
| Vitamin B₁₂ | 1 | mg |
| CaCO₃ | 30 | g |
| Yeast extract | 2 | g |

| | |
|---|---|
| pH | 7.0 |

### Example 3-1

### L-methionine Productivity of Transformed Microorganism E. coli W3110/ygaZH

The transformed microorganism E. coli W3110/ygaZH prepared in Example 2 was cultured on an LB solid medium in a 31° C. incubator overnight, and then using a platinum loop, a single colony was inoculated in a 250□ Erlenmeyer flask containing 25□ of titer medium of Table 1, followed by incubation at 31° C. and 200 rpm for 48 hrs. L-methionine from the culture broth was analyzed by HPLC, and the results are shown in Table 2.

As shown in Table 2, the pCL1920 vector was introduced into the parent strain E. coli W3110 to prepare a transformed microorganism as a control strain. In the case of culturing the control strain for 48 hrs, L-methionine of 5 □/L or less were produced. In contrast, in the case of culturing the experimental strain of the present invention (accession number KCCM10818P), which was prepared by introducing the recombinant plasmid pCL-(trc)ygaZH prepared in Example 1-3, for 48 hrs, L-methionine was produced by 90 □/L in a high yield. As a result, it can be seen that the transformed strain of the present invention produced 85 □/L more than the control.

TABLE 2

| Parent strain | E. coli W3110 | E. coli W3110 |
|---|---|---|
| Plasmid | pCL1920 | pCL-(trc)ygaZH |
| L-methionine (mg/L) | 5 or less | 90 |

### Example 3-2

### L-methionine Productivity of Transformed Microorganism E. coli MF001/ygaZH

The transformed microorganism E. coli MF001/ygaZH prepared in Example 2 was cultured on an LB solid medium in a 31° C. incubator overnight, and then using a platinum loop, a single colony was inoculated in a 250□ Erlenmeyer flask containing 25□ of titer medium of Table 1, followed by incubation at 31° C. and 200 rpm for 48 hrs. L-methionine from the culture broth was analyzed by HPLC, and the results are shown in Table 3.

As shown in Table 3, the pCL1920 vector was introduced into the parent strain E. coli MF001 to prepare a transformed microorganism as a control strain. In the case of culturing the control strain for 48 hrs, and L-threonine of 16 g/L and L-methionine of 5 □/L or less were produced. In contrast, in the case of culturing the experimental strain of the present invention E. coli MF001/ygaZH, which was prepared by introducing the recombinant plasmid pCL-(trc)ygaZH prepared in Example 1-3, for 48 hrs, L-threonine of 5.5 g/L and L-methionine of 170 □/L were produced. As a result, it can be seen that the L-threonine productivity was reduced and L-methionine productivity was increased by 165 □/L or more, as compared to the control.

TABLE 3

| Parent strain | E. coli MF001 | E. coli MF001 |
|---|---|---|
| Plasmid | pCL1920 | pCL-(trc)ygaZH |
| L-threonine (g/L) | 16.0 | 5.5 |
| L-methionine (□/L) | 5 or less | 170 |

From the results, it was found that the biosynthesis of L-methionine was improved by transforming E. coli W3110 and the non-methionine auxotrophic threonine-producing strain E. coli MF001 with the recombinant plasmid, in which the ygaZ gene encoding a hypothetical protein and the ygaH gene encoding a putative transport protein, of which functions are not clearly known, were replaced with the trc promoter. Further, it was found that the overexpressed YgaZH polypeptide significantly increased L-methionine productivity in the microorganism. The YgaZ is putatively a hypothetical transporter and the YgaH is putatively an inner membrane protein, disclosed in literature (Serres01: Serres M H, Gopal S, Nahum L A, Liang P, Gaasterland T, Riley M (2001). “A functional update of the Escherichia coli K-12 genome.” Genome Biol. 2001; 2(9); RESEARCH0035. PMID: 11574054). Further, it was found that the polypeptide is similar to brnFE that is a putative L-methionine exporter in Corynebacterium glutamicum. Accordingly, it was inferred that the YgaZH polypeptide is an L-methionine exporter in accordance with its protein structure.

It will be apparent to those skilled in the art that various modifications and changes may be made without departing from the scope and spirit of the invention. Therefore, it should be understood that the above embodiment is not limitative, but illustrative in all aspects. The scope of the invention is defined by the appended claims rather than by the description preceding them, and therefore all changes and modifications that fall within meets and bounds of the claims, or equivalents of such meets and bounds are therefore intended to be embraced by the claims.

### INDUSTRIAL APPLICABILITY

As described above, the present invention provides an YgaZ and YgaH polypeptide or a complex thereof, referred to herein as a YgaZH polypeptide, which are putative L-methionine exporters and increase the productivity of L-methionine in a microorganism, polynucleotides encoding the same, a recombinant vector comprising the polynucleotide, a microorganism transformed with the recombinant vector, and a method for producing L-methionine and/or S-adenosyl-methionine from the transformed microorganism. The microorganism transformed with the polynucleotide encoding the polypeptide according to the present invention produces L-methionine in a high yield, thereby being used for medicinal and pharmaceutical industries and feed industry, in particular, animal feeds.

1. A YgaZH polypeptide capable of increasing the production of L- methionine in a microorganism, consisting of a YgaZ polypeptide having an amino acid sequence represented by SEQ ID NO. 1 and a YgaH polypeptide having an amino acid sequence represented by SEQ ID NO. 2.

2. A polynucleotide encoding a YgaZH polypeptide capable of increasing the production of L-methionine in a microorganism consistin of a YgaZ polypeptide having an amino acid sequence represented by SEQ ID NO. 1 and a YgaH polypeptide having an amino acid sequence represented by SEQ ID NO. 2.

3. The polynucleotide according to claim 2, wherein the polynucleotide encoding the YgaZ polypeptide is represented by SEQ ID NO. 3 and the polynucleotide encoding the YgaH polypeptide is represented by SEQ ID NO. 4.

4. A recombinant vector comprising the polynucleotide of claim 2.

5. The recombinant vector according to claim 4, wherein the recombinant vector is pCL-(trc)ygaZH shown in FIG. 1.

6. A transformed microorganism having an improved productivity of L-methionine, transformed with the recombinant vector of claim 4.

7. The transformed microorganism according to claim 6, wherein the microorganismis derived from any one selected from the group consisting of Escherichia, Aerobacter, Schizosaccharomyces, Zygosaccharomyces, Pichia, Kluyveromyces, Candida, Hansenula, Debaryomyces, Mucor, Torulopsis, Methylobacter, Salmonella, Bacillus, Streptomyces, Pseudomonas and Corynebacterium sp.

8. The transformed microorganism according to claim 7, wherein the transformed microorganism is Escherichia coli.

9. The transformed microorganism according to claim 8, wherein the transformed microorganism is the one identified by accession number: KCCM 10818P.

10. A method for producing L-methionineor a derivative thereof, comprising:
(a) culturing the microorganism of claim 6, and
(b) separating L-methionine or the derivative thereof from the culture broth.

11. The method according to claim 10, wherein the L-methionine derivative is S-adenosyl-methionine.


SEQUENCE LISTING (8 sequences)

SEQ ID NO: 1 - protein, 245 aa, Escherichia coli
MESPTPQPAPGSATFMEGCKDSLPIVISYIPVAFAFGLNATRLGFSPLESVFFSCIIYAGASQFVITAMLAAGSSLWIAALTVMAMDVRHVLYGPSLRSRIIQRLQKSKTALWAFGLTDEVFAAATAKLVRNNRRWSENWMIGIAFSSWSSWVFGTVIGAFSGSGLLQGYPAVEAALGFMLPALFMSFLLASFQRKQSLCVTAALVGALAGVTLFSIPVAILAGIVCGCLTALIQAFWQGAPDEL

SEQ ID NO: 2 - protein, 111 aa, Escherichia coli
MSYEVLLLGLLVGVANYCFRYLPLRLRVGNARPTKRGAVGILLDTIGIASICALLVVSTAPEVMHDTRRFVPTLVGFAVLGASFYKTRSIIIPTLLSALAYGLAWKVMAII

SEQ ID NO: 3 - DNA, 738 bp, Escherichia coli
atggaaagccctactccacagcctgctcctggttcggcgaccttcatggaaggatgcaaagacagtttaccgattgttattagttatattccggtggcctttgcgttcggtctgaatgcgacccgtctgggattctctcctctcgaaagcgtttttttctcctgcatcatttatgcaggcgcgagccagttcgtcattaccgcgatgctggcagccgggagtagtttgtggattgctgcactgaccgtcatggcaatggatgttcgccatgtgttgtatggcccgtcactgcgtagccgtattattcagcgtctgcaaaaatcgaaaaccgccctgtgggcgtttggcctgacggatgaggtttttgccgccgcaaccgcaaaactggtacgcaataatcgccgctggagcgagaactggatgatcggcattgccttcagttcatggtcatcgtgggtatttggtacggtaataggggcattctccggcagcggcttgctgcaaggttatcccgccgttgaagctgcattaggttttatgcttccggcactctttatgagtttcctgctcgcctctttccagcgcaaacaatctctttgcgttaccgcagcgttagttggtgcccttgcaggcgtaacgctattttctattcccgtcgccattctggcaggcattgtctgtggctgcctcactgcgttaatccaggcattctggcaaggagcgcccgatgagctatga

SEQ ID NO: 4 - DNA, 336 bp, Escherichia coli
atgagctatgaggttctgctgcttgggttactagttggcgtggcgaattattgcttccgctatttgccgctgcgcctgcgtgtgggtaatgcccgcccaaccaaacgtggcgcggtaggtattttgctcgacaccattggcatcgcctcgatatgcgctctgctggttgtctctaccgcaccagaagtgatgcacgatacacgccgtttcgtgcccacgctggtcggcttcgcggtactgggtgccagtttctataaaacacgcagcattatcatcccaacactgcttagtgcgctggcctatgggctcgcctggaaagtgatggcgattatataa

SEQ ID NO: 5 - DNA, 24 bp, Artificial Sequence
  forward primer for ygaZH amplification from Escherichia coli
ccatggaaagccctactccacagc

SEQ ID NO: 6 - DNA, 24 bp, Artificial Sequence
  reverse primer for ygaZH amplification from Escherichia coli
gagctcttatataatcgccatcac

SEQ ID NO: 7 - DNA, 34 bp, Artificial Sequence
  forward primer for amplification of (trc)ygaZH
caagtgacggggtaccttgcgccgacatcataac

SEQ ID NO: 8 - DNA, 34 bp, Artificial Sequence
  reverse primer for amplification of (trc)ygaZH
caagtgatgctctagacaaaaaggccatccgtca